(19) United States
(12) Patent Application Publication
(10) Pub. No.: US 2012/0123332 A1
(43) Pub. Date: May 17, 2012

(54) NEEDLE-BASED MEDICAL DEVICE AND RELATED METHOD

(75) Inventor: Timothy J. Erskine, Sister Bay, WI (US)

(73) Assignee: ERSKINE MEDICAL LLC, High Falls, NY (US)

(21) Appl. No.: 13/254,163

(22) PCT Filed: Mar. 5, 2009
(86) PCT No.: PCT/US09/36197

§ 371 Date: Aug. 31, 2011

(51) Int. Cl. A61M 5/50; B23P 17/04

(52) U.S. Cl. 604/110; 29/428

(57) ABSTRACT

A needle-based medical device and a method for constructing it are disclosed. The medical device includes a needle blocking object movable from a non-blocking position offset from the longitudinal axis of the needle to a blocking position; a needle shield housing, the needle shield housing including: a lumen for accommodating at least part of the needle such that the needle shield housing; an opening for accommodating at least part of the needle blocking object; and a biasing member for applying a biasing force on the needle blocking object, the biasing force including a first component substantially perpendicular to the longitudinal axis of the needle and a second component substantially parallel to the longitudinal axis of the needle. The needle blocking object moves by exertion of the biasing force to the blocking position, preventing the needle from emerging from the needle shield housing, in response to a triggering proximal movement of the needle.

### TECHNICAL FIELD

The disclosure relates generally to a needle-based medical device, and more particularly, to a needle-based medical device having a needle blocking feature.

### BACKGROUND ART

Huber needles are used in the medical industry to access implanted ports provided in patients for long-term drug therapy. Huber needles are structured to minimize coring which occurs with a conventional needle when it is inserted into the elastomeric septum and a portion of the septum is cut away. Huber needles typically include a right angle needle which extends from a hub, and some sort of horizontally planar member such as coplanar wings that extend outwardly from the hub. The horizontally planar member is used to grasp and manipulate the Huber needle during accessing of the implanted ports. A right angle needle is used because, due to the perpendicular nature of the needle's insertion, any movement of the needle can cause discomfort to the patient. The right angle needle minimizes the overall height and, consequently, the amount of the needle disposed away from the patient's skin that may be inadvertently moved.

As in the case of hypodermic and intravenous needles, the safety of Huber needles is of great concern. To address this concern, safety devices in the form of needle shields are used to prevent accidental needle sticks, thus minimizing the risk of the transmission of infectious diseases. The shielding of Huber needles pose some technological challenges in that the patient must be provided with a high level of comfort by minimizing the overall height of the device.

### SUMMARY OF THE INVENTION

A needle-based medical device and a related method for constructing the same are disclosed. Embodiments of the needle-based medical device include: a needle having a proximal end, a sharp distal end and a longitudinal axis; a needle blocking object movable from a non-blocking position offset from the longitudinal axis of the needle to a blocking position; a needle shield housing, the needle shield housing including: a lumen for accommodating at least part of the needle such that the needle shield housing is slidable at least partially along the longitudinal axis of the needle; an opening for accommodating at least part of the needle blocking object in the non-blocking position; and a biasing member for applying a biasing force on the needle blocking object, the biasing force including a first component substantially perpendicular to the longitudinal axis of the needle and a second component substantially parallel to the longitudinal axis of the needle, wherein the needle blocking object moves by exertion of the biasing force to the blocking position, preventing the needle from emerging from the needle shield housing, in response to a triggering proximal movement of the needle.

Embodiments of the method may include: placing a needle shield housing around a needle such that a lumen of the needle shield housing surrounds at least a part of the needle; positioning a needle blocking object at least partially within an opening in the needle shield in a non-blocking position; positioning a biasing member for applying a biasing force on the needle blocking object, the biasing force including a first component substantially perpendicular to the longitudinal axis of the needle and a second component substantially parallel to the longitudinal axis of the needle; and attaching an enclosure to the needle shield housing.

Further embodiments of a medical device may include a needle having a proximal end, a sharp distal end and a longitudinal axis; a ball bearing movable from a non-blocking position offset from the longitudinal axis of the needle to a blocking position; a needle shield housing, the needle shield housing including: a lumen for accommodating at least part of the needle such that the needle shield housing is slidable at least partially along the longitudinal axis of the needle; an opening for accommodating at least part of the ball bearing in the non-blocking position; and a biasing member for applying a biasing force on the ball bearing, the biasing force including a first component substantially perpendicular to the longitudinal axis of the needle and a second component substantially parallel to the longitudinal axis of the needle, wherein the ball bearing moves by exertion of the biasing force to the blocking position, preventing the needle from emerging from the needle shield housing, in response to a triggering proximal movement of the needle, and wherein the ball bearing is retained in the blocking position by the biasing force.

The illustrative aspects of the invention are designed to solve one or more of the problems herein described and/or one or more other problems not discussed.

### BRIEF DESCRIPTION OF THE DRAWINGS

These and other features of the disclosure will be more readily understood from the following detailed description of the various aspects of the invention taken in conjunction with the accompanying drawings that depict various aspects of the invention.

FIG. 1 shows a cross-sectional view of a needle-based medical device (a Huber needle) according to one embodiment of the invention in a non-blocking position.

FIG. 2 shows a cross-sectional view of a needle-based medical device (a Huber needle) according to one embodiment of the invention in a blocking position.

FIG. 3 shows a side perspective view of a needle shield housing.

FIG. 4 shows a cross-sectional view of the needle shield housing in a non-blocking position.

FIG. 5 shows a cross-sectional view of the needle shield housing in a blocking position.

FIG. 6 shows a side perspective view of a telescopic member.

FIGS. 7A-G show views of alternative embodiments of a biasing member and the needle shield housing.

FIG. 8 shows a cross-sectional view of an alternative embodiment of the needle-based medical device (Huber needle).

FIG. 9 shows a cross-sectional view of an alternative embodiment of the needle-based medical device (Huber needle) in a non-blocking position.

FIG. 10 shows a a cross-sectional view of an alternative embodiment of the needle-based medical device (Huber needle) in a blocking position.

FIG. 11 shows a perspective view of the needle shield housing for the FIGS. 9 and 10 embodiment.

FIGS. 12-14 show cross-sectional views of the needle shield housing of FIGS. 9 and 10 in the non-blocking position, during transition and in the blocking position, respectively.

FIG. 15 shows a cross-sectional view of an alternative embodiment of the needle-based medical device (Huber needle) in the non-blocking position.

FIG. 16 shows a cross-sectional view of an alternative embodiment of the needle-based medical device (Huber needle) in the blocking position.

FIG. 17 shows a cross-sectional view of an alternative embodiment of a needle shield housing in the non-blocking position.

FIG. 18 shows a cross-sectional view of the needle shield housing of FIG. 17 in the blocking position.

FIGS. 19-27 show perspective views of embodiments of a method of constructing the needle-based medical device (Huber needle).

It is noted that the drawings are not to scale. The drawings are intended to depict only typical aspects of the invention, and therefore should not be considered as limiting the scope of the invention. In the drawings, like numbering represents like elements between the drawings.

### DETAILED DESCRIPTION OF THE INVENTION

As indicated above, aspects of the invention provide embodiments of a needle-based medical device and method of constructing the same.

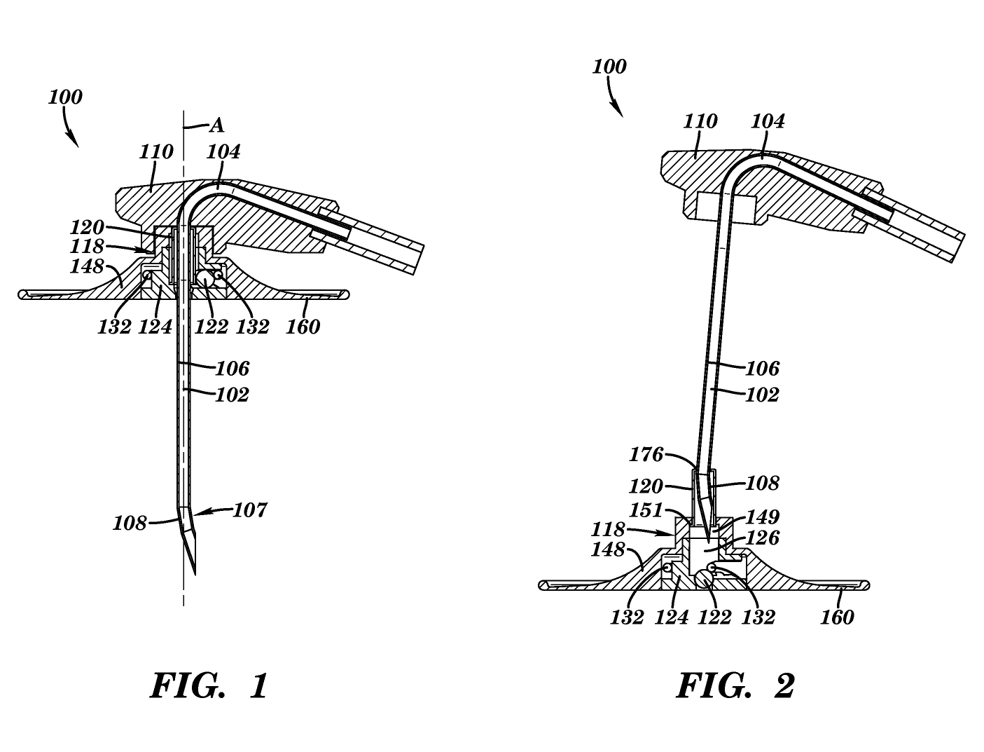

Referring now to FIGS. 1-2, one embodiment of a needle-based medical device 100 is shown including a needle 102 in the form of a Huber needle. FIG. 1 shows medical device 100 in a non-blocking position of needle 102, and FIG. 2 shows medical device 100 in a blocking position of needle 102, i.e., after use of the needle. Needle 102 includes an approximate right angle 104 to minimize the height of the needle to provide the patient with a high level of comfort during use. Needle 102 includes a proximal end 106, a sharp distal end 108 and a longitudinal axis A (FIG. 1 only). In one embodiment, sharp distal end 108 includes a stopping feature 107 formed by a localized change in the geometry of needle 102. In this embodiment, stopping feature 107 is naturally formed due to the Huber needle tip-forming process where a slight angle is bent into sharp distal end 108 of needle 102 to make the point non-coring. However, a crimp, bump or other structure capable of providing a stop to prevent the distal movement of an encircling part, but not interfere with a patient's comfort during use, may be employed. A hub 110 encloses an approximate right angle 104 of needle 102 for handling by a user. While needle 102 is illustrated herein as a Huber needle, it is also understood that needle 102 may take the form of other now known or later developed needles. For example, practically any needle with an enlarged diameter at or near sharp distal end 108 such as a crimp or circumferential bump may also work.

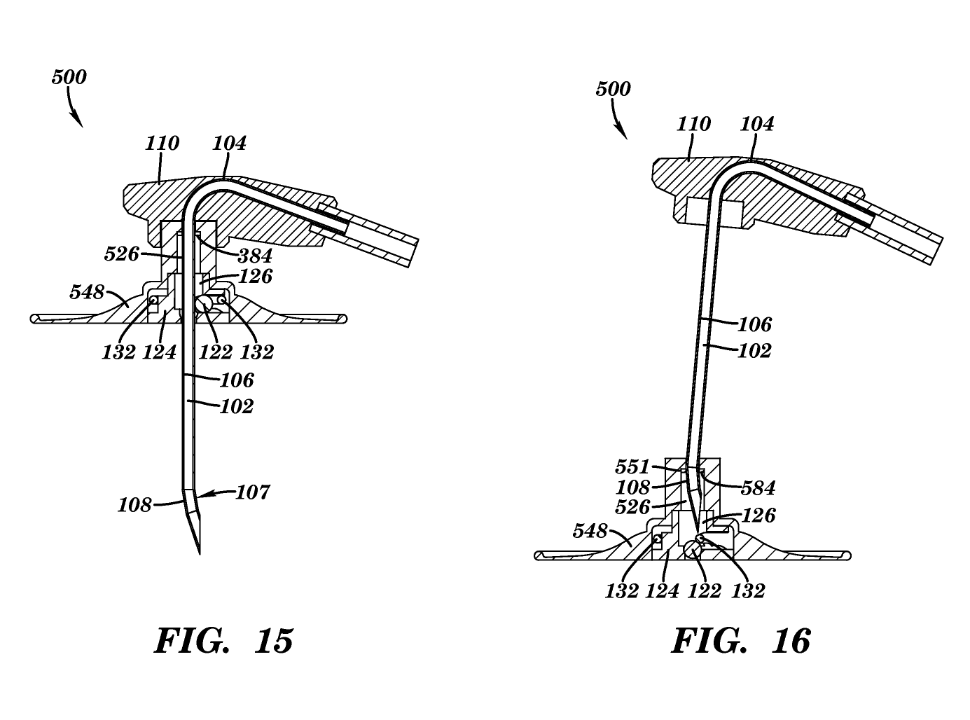

Needle based medical device 100 also includes an attachment and safety assembly 118. (The embodiments shown in FIGS. 1-5, 8-10, 12-14, 17 and 18 include a telescoping member 120, the purpose of which will be described further herein, while the FIGS. 15-16 embodiments omit the telescoping member.) Assembly 118 includes a needle blocking object 122 positioned within a needle shield housing 124. Needle blocking object 122 is movable from the non-blocking position offset from the longitudinal axis of needle 102, as shown in FIG. 1, to a blocking position, as shown in FIG. 2. As will be described in greater detail herein, in the blocking position, sharp distal end 108 of needle 102 cannot emerge from needle shield housing 124 due to needle blocking object 122 blocking its path. In one embodiment, needle blocking object 122 takes the form of a ball bearing; however, other shapes may also be possible, such as a cylindrical rod.

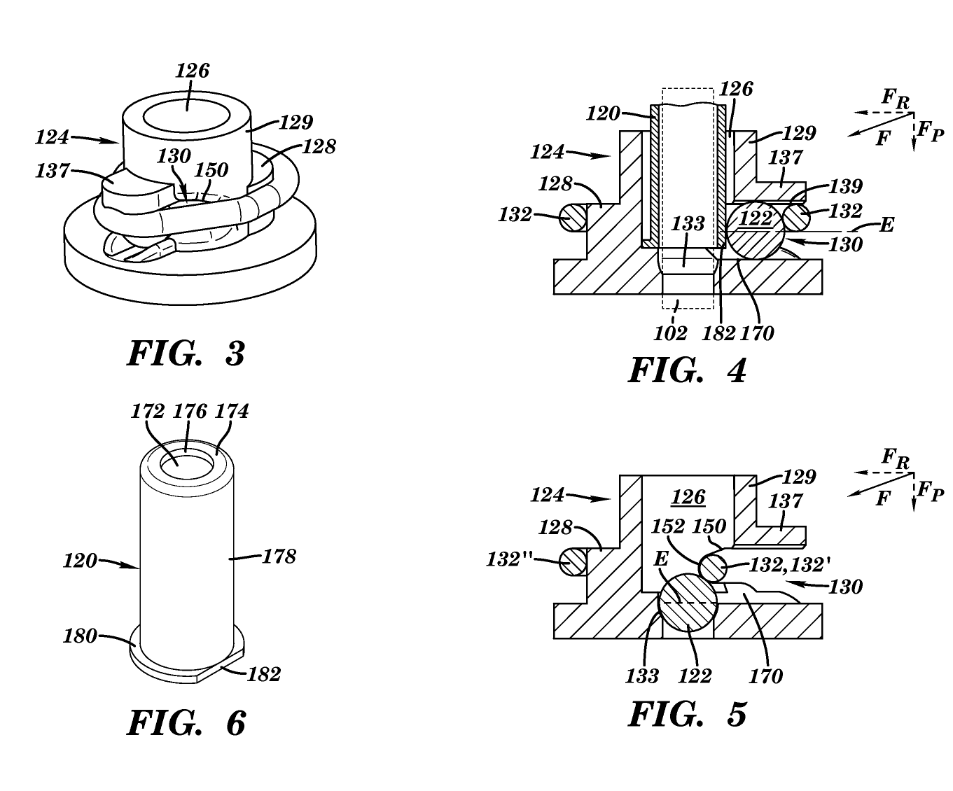

Referring to FIGS. 3-6, in conjunction with FIGS. 1 and 2, details of needle blocking object 122 and needle shield housing 124 will now be described. FIG. 3 shows an enlarged perspective view of needle shield housing 124, FIG. 4 shows an enlarged cross-sectional view of needle shield housing 124 in the non-blocking position corresponding to FIG. 1, and FIG. 5 shows an enlarged cross-sectional view of needle shield housing 124 in the blocking position corresponding to FIG. 2. FIG. 6 shows a perspective view of a telescoping member 120.

As shown best in FIGS. 3-5, needle shield housing 124 includes a lumen 126 for accommodating at least part of needle 102 (in FIG. 4 only, in phantom) such that the needle shield housing is slidable at least partially along longitudinal axis A (FIG. 1) of the needle when needle blocking object 122 is in the non-blocking position (FIG. 4). Needle shield housing 124 also includes an opening 130 for accommodating at least part of needle blocking object 122 in the non-blocking position (FIG. 4). In one embodiment, opening 130 extends through a wall of a cylindrical member 129 that forms lumen 126; however, this may not be necessary or desirable in all cases. In a non-blocking position, as shown in FIG. 4, needle blocking object 122 may be positioned at least partially within lumen 126 through a wall of cylindrical member 129 that forms lumen 126. However, this is not necessary in all cases, i.e., needle blocking object 122 need not enter lumen 126. As illustrated, opening 130 is configured to direct needle blocking object 122 such that it moves (rolls or slides) or is guided by guiding surface 170 substantially perpendicular to the longitudinal axis of needle 102 during movement between the non-blocking position (FIG. 4) and the blocking position (FIG. 5). However, in alternative embodiments, opening 130 may be angled towards sharp distal end 108 of needle 102 in the non-blocking position. As shown in FIGS. 4 and 5, needle shield housing 124 also includes a nest 133 for supporting needle blocking object 122 in the blocking position. Nest 133 includes an area configured to receive a portion of needle blocking object 122, e.g., a smaller diameter opening compared to lumen 126.

In order to move needle blocking object 122 to the blocking position, as shown best in FIG. 4, a biasing member 132 applies a biasing force F on needle blocking object 122. Biasing member 132 is a separate structure from needle blocking object 122 and may take a variety of forms. As shown in FIGS. 1-2 and 4-5, biasing member 132 includes a generally circular elastic member such as an O-ring. In this case, biasing member 132 substantially surrounds needle 102 and needle shield housing 124, and is on the outside of the needle shield housing. As a circular elastic member, biasing member 132 may be substantially coaxial with longitudinal axis A (FIG. 1). In order to optimize the strain on biasing member 132, needle shield housing 124 may include an offset portion 128 for stretching the biasing member. As illustrated, offset portion 128 includes a cam; however, other structures may be employed. Offset portion 128 may be a separate structural member, or as illustrated, may be provided as an area of enlarged radius of cylindrical member 129 that forms lumen 126. In addition to cam 128, needle shield housing 124 may also include a flange 137 for properly positioning biasing member 132 relative to needle blocking object 122. In one embodiment, flange 137 may include a track 139 for accommodating needle blocking object 132; however, this may not be necessary in all cases.

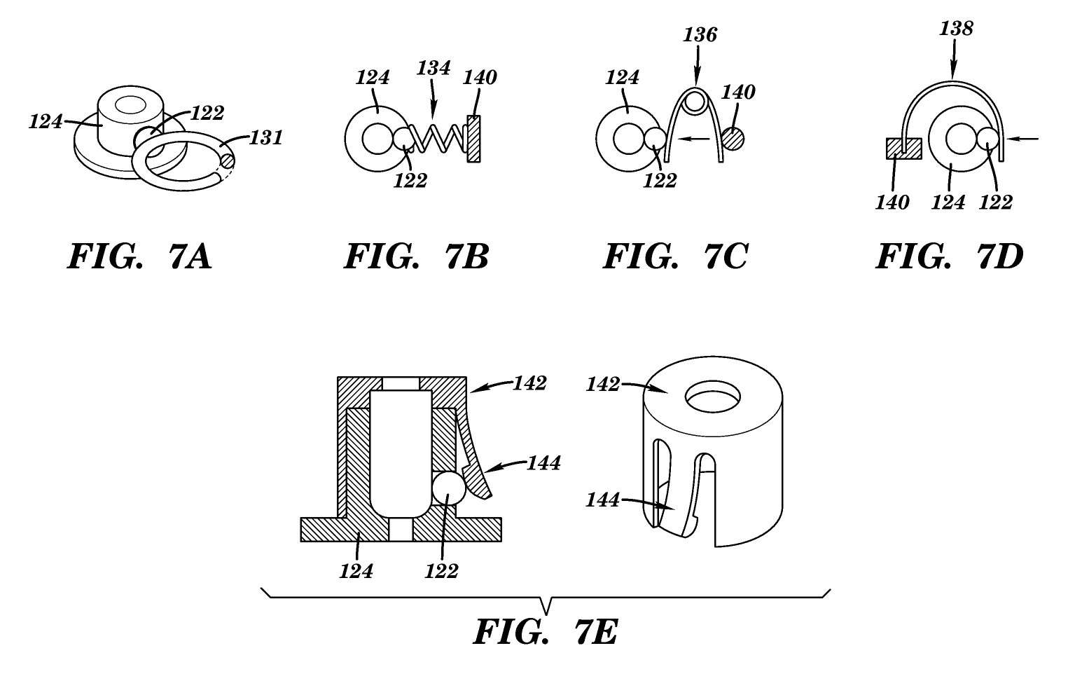

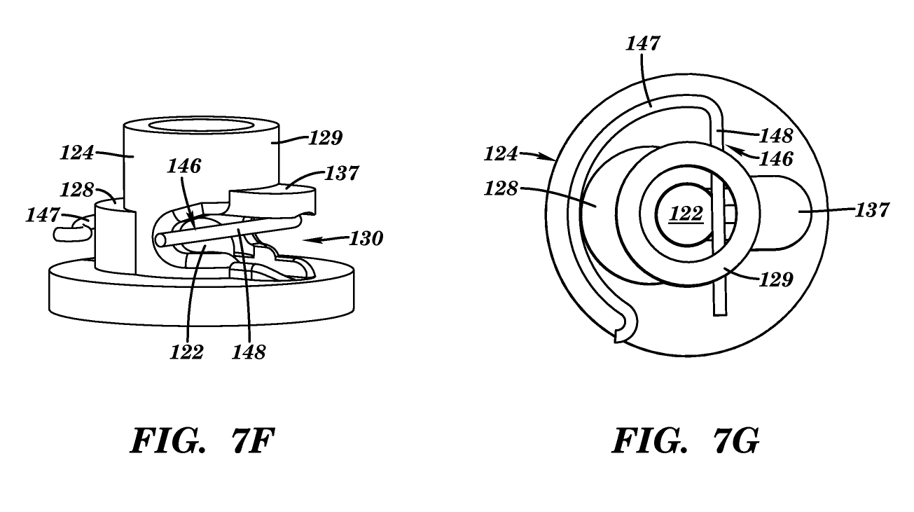

FIGS. 7A-7G show illustrative alternative embodiments of a biasing member which include, for example, an offset O-ring 131 (FIG. 7A) that expands to press against needle blocking object 122, a compression spring 134 (FIG. 7B), a torsion spring 136 (FIG. 7C) and a spring wire 138 (FIG. 7D), each of which may require a fixing member 140 for proper positioning. FIG. 7E shows another embodiment in which the biasing member includes a molded latch 142 having a spring latch 144 for exerting the biasing force against needle blocking object 122. Certain of the above-described embodiments may be formed such that they are entirely within needle shield housing 124. FIGS. 7F-7G show another embodiment in which the biasing member includes a D-shaped retainer 146 for exerting the biasing force against needle blocking object 122. A semi-circular portion 147 of D-shaped retainer 146 engages cam 128, and a straight portion 148 of D-shaped retainer engages needle blocking object 122. FIG. 7F shows a perspective view in the blocking position, and FIG. 7G shows a plan view in the blocking position.

Returning to FIG. 4, regardless of the biasing member used, biasing force F includes a first component F_{R} substantially perpendicular to longitudinal axis A (FIG. 1) of needle 102 (or radially from the longitudinal axis) and a second component F_{P} substantially parallel to the longitudinal axis of the needle. In the FIGS. 1-5 embodiments, where needle blocking object 122 includes a ball bearing with an equator E (shown as extended dashed line in FIG. 4), biasing member 132 exerts biasing force F by making contact with the ball bearing on a proximal side of equator E. Although illustrated as such, biasing member 132 need not be entirely proximal of equator E, so long at its position relative to needle blocking object 122 is maintained to direct biasing force F as described above. Similar application of the force can be created using any of the embodiments illustrated in FIGS. 7A-7G.

As shown best in FIGS. 3 and 5, needle shield housing 124 also includes a guide 150 such as a bearing surface or other structure to substantially maintain biasing member 132 on needle blocking object 122 to retain biasing force F with the above described components. More particularly, as observed in FIG. 5, as needle blocking object 122 moves to the blocking position, it moves distally. Guide 150 ensures that while the contact point may shift, biasing member 132 maintains its position relative to needle blocking object 122 such that biasing force F components F_{R} and F_{P} continue in force. In the embodiment shown, guide 150 is in communication with opening 130; however, separate structure may also be employed externally of the opening, if desired, and a gap or other structure may be interposed between them. Guide 150 may also include a recessed portion 152 (FIG. 5) (relative to an inner diameter of lumen 126) for receiving at least part of biasing member 132. In this manner at least one part 132′ of biasing member 132 is closer to the longitudinal axis of lumen 126 than another part 132″ of the biasing member in the blocking position. Furthermore, guide 150 in the form of a bearing surface may be at practically any angle necessary to properly position biasing member 132, e.g., at least partially: substantially perpendicular, at an acute angle or at an obtuse angle relative to the longitudinal axis A (FIG. 1).

Returning to FIGS. 1 and 2, an enclosure 148 covers needle shield housing 124. In the FIGS. 1 and 2 embodiments, enclosure 148 substantially surrounds needle shield housing 124 and includes an integral disk 160 that extends away from the needle shield housing. Disk 160 may be employed to manipulate needle 102 during use and/or secure medical device 100 to a patient, e.g., using tape or other adhesives, and may comprise grasping wings. Enclosure 148 may be made of a semi-rigid material, so that it is flexible when thin (i.e., at disk 160) and rigid when thicker (i.e., adjacent housing 124). The material for enclosure 148 may include polypropylene, polyethylene, Acetal® or other semi-rigid materials. As shown best in FIG. 2, enclosure 148 may also include a lumen 149 having a smaller diameter end flange 151. Lumen 149 may be configured to match a diameter of lumen 126.

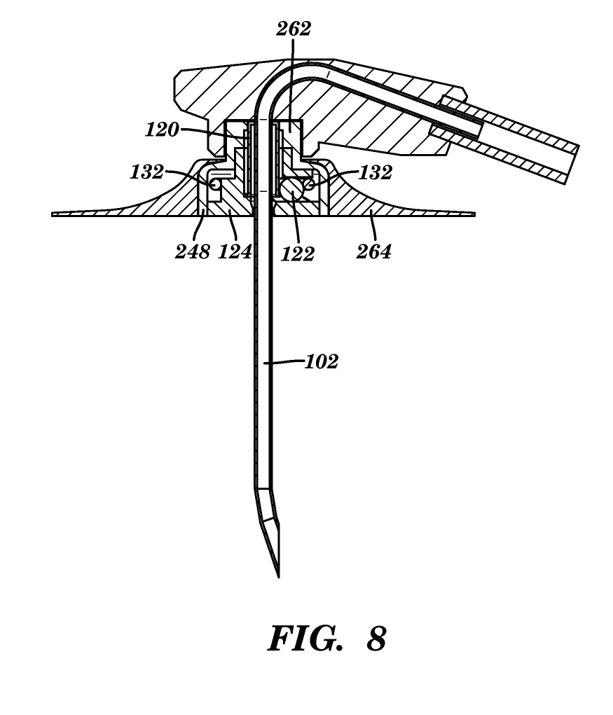

FIG. 8 shows an alternative embodiment of an enclosure 248 for needle shield housing 124. In this case, an inner portion 262 of enclosure 248 may include a rigid material such as polystyrene and polycarbonate and an outer portion 264 may include semi-rigid materials, as discussed above, such as polypropylene, polyethylene, and Acetal®, or soft, flexible material such as Thermoplastic Elastomer (TPE) or silicone. Inner portion 262 encloses needle shield housing 124 including biasing member 132, and outer portion 264 is coupled to inner portion 262, e.g., by interference fit, snap fit, adhesive, etc.

In the FIGS. 1-5 and 8 embodiments, as shown best in FIG. 4, telescoping member 120 is slidably positioned within lumen 126 and holds needle blocking object 122 in the non-blocking position. Telescopic member 120 may be formed by any now known or later developed means, for example, by metal stamping out of, for example, stainless steel about 0.006 inches thick. As shown in FIG. 6, telescopic member 120 includes a lumen 172 having a diameter sufficiently large so as to allow unencumbered sliding movement of needle 102 therethrough, including sharp distal end 108 and stopping feature 107 (FIG. 1), e.g., a bend. However, telescopic member 120 also includes a first end 174 having a lumen 176 having a smaller diameter than lumen 172. Sharp distal end 108 of needle 102, i.e., stopping feature 107, cannot pass through lumen 176. That is, stopping feature 107 (FIG. 1) of sharp distal end 108 prevents needle 102 from separating from telescoping member 120. The purpose of this structure will be described elsewhere herein. With continuing reference to FIG. 6, a second end 178 of telescoping member 120 may include a flange 180 thereabout that aids in positioning telescoping member 120 substantially concentrically within lumen 126 of needle shield housing 124. Further, as shown in FIG. 2, flange 180 (not shown) abuts end flange 151 of enclosure 148, 248 to prevent removal of telescoping member 120 from the enclosure in the blocking position. As shown in FIG. 6, flange 180, however, includes a cut away 182 on a side thereof. As shown in FIG. 4, cut away 182 faces needle blocking object 122. Cut away 182 allows telescopic member 120 to pass needle blocking object 122 during proximal movement thereof without interfering with the needle blocking object, i.e., without snagging or catching on needle blocking object 122 sufficiently to stop movement of the telescopic member. It is emphasized, however, that cut away 182 may not be necessary in cases where flange 180 can pass needle blocking object 122 without snagging or otherwise causing excessive interference with needle blocking object 122.

Referring to FIGS. 1, 2, 4 and 5, operation of needle-based medical device 100 will now be described. In operation, needle blocking object 122 moves by exertion of biasing force F to the blocking position (FIGS. 2 and 5), preventing needle 102 from emerging from needle shield housing 124, in response to a triggering proximal movement of the needle. In the FIGS. 1, 2, 4 and 5 embodiments, a triggering proximal movement of the needle occurs when needle 102 moves proximally relative to needle shielding housing 124 sufficiently to engage telescopic member 120 and move telescopic member 120 out of contact with needle blocking object 122. In particular, referring to FIGS. 2 and 6, as needle 102 moves proximally, the stopping feature 107 (FIG. 1) of sharp distal end 108 engages lumen 176 of telescoping member 120. As this occurs, telescoping member 120 extends longitudinally relative to lumen 126 of needle shield housing 124. This movement of telescoping member 120 has two effects. First, it enlarges the volume of lumen 126 and thus needle shield housing 124 so as to accommodate sharp distal end 108, which allows for safety protection without having to have a taller medical device. Second, as telescoping member 120 moves, it passes needle blocking object 122, perhaps with the aid of cut away 182, and eventually loses contact with needle blocking object 122. The telescopic movement of telescoping member 120 continues until flange 180 thereof engages end flange 151 of enclosure 148 which prevents separation of telescoping member 120 from enclosure 148 and needle shield housing 124. As noted above, stopping feature 107 (FIG. 1) of sharp distal end 108 prevents needle 102 from separating from telescoping member 120.

Once this triggering proximal movement occurs, needle blocking object 122 moves freely to the blocking position, shown in FIGS. 2 and 5, under the influence of biasing force F. More specifically, once telescopic member 120 passes needle blocking object 122, the needle blocking object initially moves substantially perpendicular to the longitudinal axis of needle 102 as it moves along guiding surface 170 (FIGS. 4-5) of opening 130. Needle blocking object 122 is forced towards lumen 126 by the substantially perpendicular force F_{R} (relative to longitudinal axis A (FIG. 1)) of biasing force F and is held in contact with guiding surface 170 by substantially parallel component F_{P} (relative to longitudinal axis A) of biasing force F. As shown in FIGS. 2 and 5, once guiding surface 170 can no longer guide needle blocking object 122 towards lumen 126, the needle blocking object is moved distally by the substantially parallel force F_{P} and into nest 133, where it is held under the influence of biasing force F. At this point, as shown in FIG. 2, sharp distal end 108 of needle 102 is positioned within lumen 126 and/or telescoping member 120, and cannot move distally to emerge from needle shield housing 124 because needle blocking object 122 blocks its path. During transition from the non-blocking position (FIG. 4) to the blocking position (FIG. 5), biasing member 132 is guided by guide 150 so as to maintain contact with needle blocking object 122 and thus maintain the appropriate direction of biasing force F.

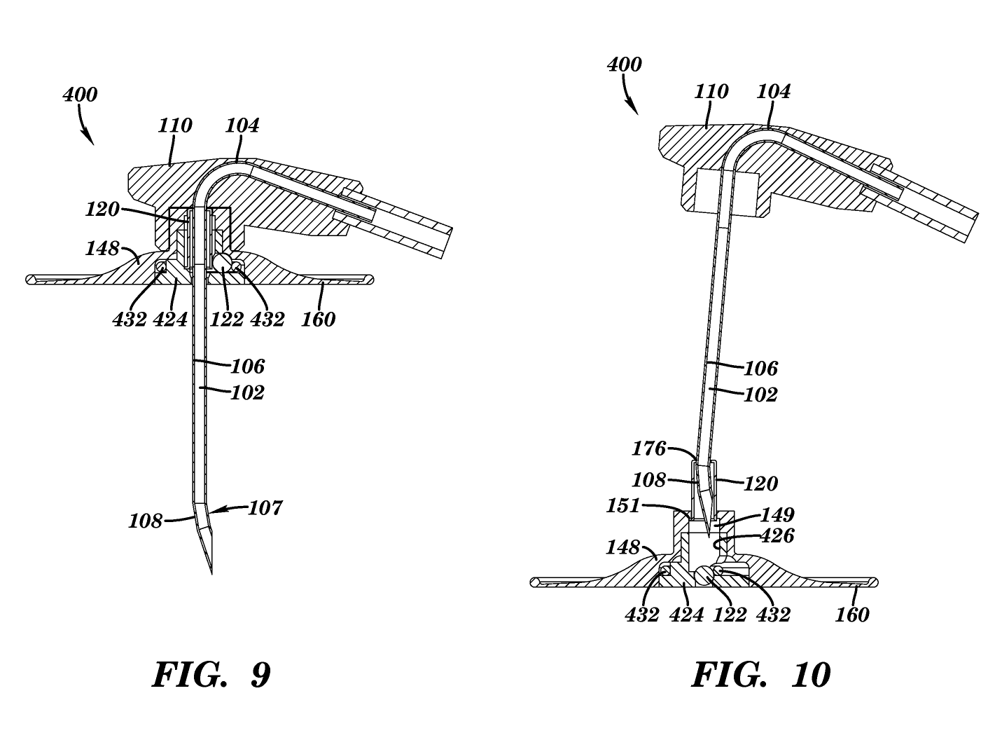

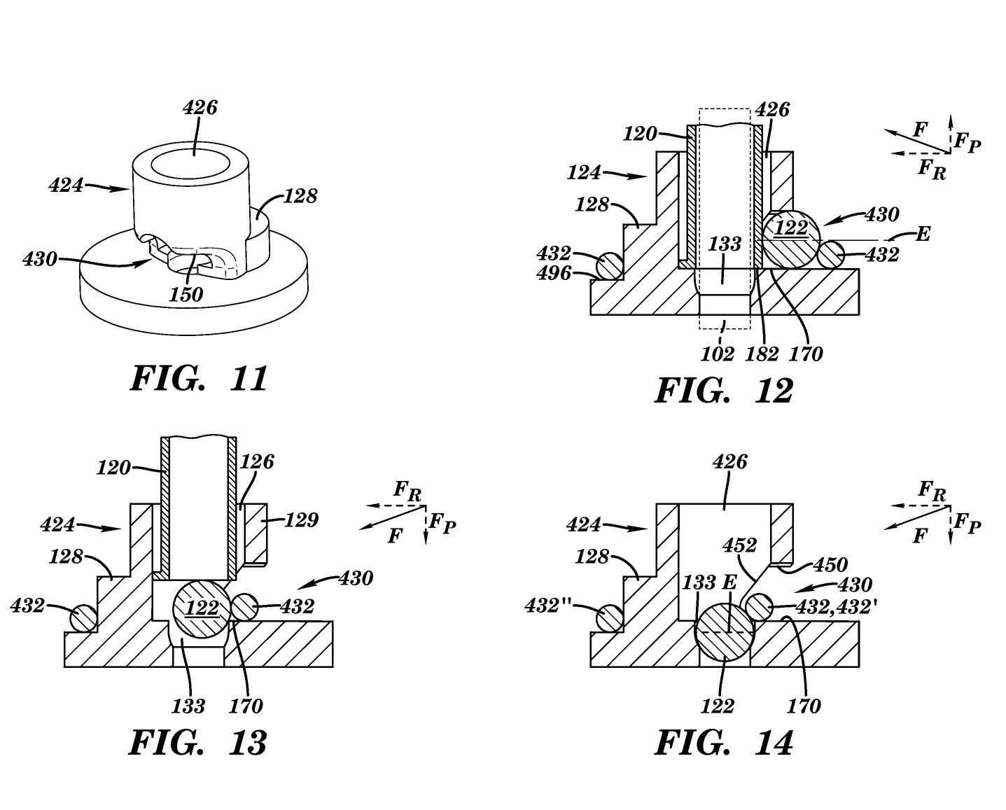

Referring to FIGS. 9-14, an alternative embodiment of a needle-based medical device 400 is illustrated. This embodiment is substantially similar to that shown for FIGS. 1-5, except that a needle shield housing 424 in this embodiment is structured such that a biasing member 432 is disposed such that biasing force F is exerted on a distal side of equator E of needle blocking object 122 in the non-blocking position (FIGS. 9 and 12) and transitions to exert the biasing force on a proximal side of the equator during transitioning (FIG. 13) from the non-blocking position to the blocking position (FIG. 14). In this case, flange 137 (FIG. 4-5) may be omitted, as shown best in FIG. 11, and biasing member 432 and/or enclosure 148 may be all that is needed to position needle blocking object 122 in the non-blocking position. Biasing member 432 may be positioned on the distal side of the equator E by lowering a surface 496 (FIG. 12) upon which biasing member 432 is positioned next to cam 128 to be substantially planar or only slightly higher than guiding surface 170. This is in contrast to the FIGS. 1-5 embodiments in which that surface is substantially higher than guiding surface 170.

Referring to FIGS. 9 and 10 in conjunction with FIGS. 12-14, operation of the needle-based medical device will now be described. In operation, needle blocking object 122 moves by exertion of biasing force F to the blocking position (FIGS. 10 and 14), preventing needle 102 from emerging from needle shield housing 424, in response to a triggering proximal movement of the needle. In the FIGS. 9 and 10 embodiments, a triggering proximal movement of needle 102 occurs when the needle moves proximally relative to needle shielding housing 424 sufficiently to engage telescopic member 120 and move telescopic member 120 out of contact with needle blocking object 122. In particular, referring to FIGS. 10 and 14, as needle 102 moves proximally, stopping feature 107 (FIG. 9) of sharp distal end 108 engages lumen 176 (FIG. 6) of telescoping member 120. As this occurs, telescoping member 120 extends longitudinally relative to lumen 426 of needle shield housing 424. This movement of telescoping member 120 has three effects. First, it enlarges the volume of lumen 426 and thus needle shield housing 424 so as to accommodate sharp distal end 108, which allows for safety protection without having to have a taller medical device. Second, as telescoping member 120 moves, it passes needle blocking object 122, perhaps with the aid of cut away 182, and eventually loses contact with needle blocking object 122. Third, in contrast to other embodiments, as shown in FIG. 13, as telescoping member 120 passes needle blocking object 122 and the object transitions from the non-blocking position (FIG. 12) to the blocking position (FIG. 14), telescoping member 120 slows progress of needle blocking object 122 such that biasing member 432 changes a point of contact therewith from being on the distal side of equator E (FIG. 14) of the object to the proximal side of equator E (FIG. 14). This transition acts to change the direction of the substantially parallel force F_{P} (relative to longitudinal axis A (FIG. 1 only)) from acting proximally (FIG. 12) to acting distally (FIG. 13-14). The telescopic movement of telescoping member 120 continues until flange 180 thereof engages end flange 151 of enclosure 148, which prevents separation of telescoping member 120 from enclosure 148 and needle shield housing 424.

Once this triggering proximal movement occurs, needle blocking object 122 moves freely to the blocking position, shown in FIGS. 10 and 14, under the influence of biasing force F. More specifically, once telescopic member 120 passes needle blocking object, needle blocking object 122 initially moves substantially perpendicular to the longitudinal axis of needle 102 as it moves along a guiding surface 170 (FIGS. 12-14) of opening 130. Needle blocking object 122 is forced towards lumen 426 by the substantially perpendicular force F_{R} of biasing force F and is held in contact with guiding surface 170 by contact with telescoping member 120 and opening 130 until the substantially parallel component F_{P} of biasing force F transitions to a proximal side of equator E. After this occurs, component F_{P} acts to maintain contact between needle blocking object 122 and guiding surface 170. As shown in FIGS. 10, 13 and 14, once guiding surface 170 can no longer guide needle blocking object 122 towards lumen 126, the needle blocking object is moved distally by substantially parallel force F_{P} and into nest 133, where it is held under the influence of biasing force F. Biasing member 432 may further transition towards the proximal side of equator E as this occurs. At this point, sharp distal end 108 of needle 102 is positioned within lumen 426 and/or telescoping member 120, and cannot move distally to emerge from needle shield housing 424. During transition from the non-blocking position (FIG. 12) to the blocking position (FIG. 16), biasing member 432 may also be guided by a guide 450 such as a bearing surface so as to maintain contact with needle blocking object 122 and thus maintain the appropriate direction of biasing force F. A recess 452 may be provided to allow one side of biasing member 432′ to be closer to the longitudinal axis than another side 432″.

Referring to FIGS. 15 and 16, another alternative embodiment of a needle-based medical device 500 is illustrated. The structure of this medical device is substantially similar to that of medical device 100 (FIGS. 1-5) and 400 (FIGS. 9-14) except that the telescopic member is omitted. (The description hereafter will only reference the FIGS. 1-5 embodiment for clarity). In this case, in order to provide the requisite room for sharp distal end 108 of needle 102, an enclosure 548 that encloses needle shield housing 124, similarly to that described above, includes a lumen 526 that acts as an extension of lumen 126. This embodiment thus results in a taller medical device 500 than those using the telescopic member. Just as in the above-described embodiment, in operation, needle blocking object 122 moves by exertion of biasing force F to the blocking position (FIG. 4), preventing needle 102 from emerging from needle shield housing 124, in response to a triggering proximal movement of the needle. In this case, however, rather than telescoping member 120 releasing needle blocking object 122, the triggering proximal movement includes movement of sharp distal end 108 of the needle out of contact with needle blocking object 122. Needle 102 may include a stopping feature 107 (such as bend or other change in needle geometry) adapted to abut an end flange 551 of enclosure 548 to prevent proximal movement of the needle shield housing when the needle blocking object is in the blocking position. In one embodiment, a stop-washer 584, made of, for example, stainless steel about 0.010 inches thick, may be used to strengthen end flange 551 to retain needle 102 in enclosure 548 (FIG. 14). That is, stop-washer 584 prevents complete removal of needle 102 from lumen 526 of enclosure 548.

Stop-washer 584 may also be employed with the FIGS. 1-5 and 8-14 embodiments, where necessary. Also, while FIGS. 1-5 and 9-14 have been illustrated with enclosure 148 over needle shield housing 124, enclosure 248 (FIG. 8) or enclosure 548 (FIGS. 15 and 16) may also be employed with those embodiments of the respective needle shield housings.

With further regard to biasing force F, in any of the above-described embodiments, components F_{P} and F_{R} are directly applied to needle blocking object 122, 622A/622B by the respective biasing member. That is, while the needle blocking object may be guided by a guiding surface 170, components F_{P} and F_{R} are created by the direct impact of the respective biasing member on the needle blocking object, and not as a result of a reaction of the needle blocking object on the surface under the influence of uni-directional biasing force.

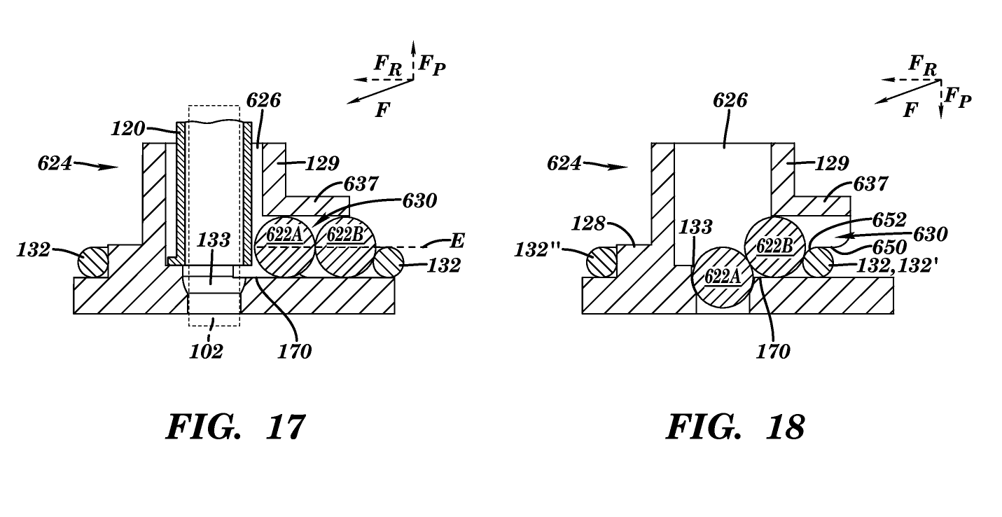

Referring to FIGS. 17 and 18, cross-sectional views of an alternative embodiment of a needle shield housing 624 are illustrated. Needle shield housing 624 is substantially similar to needle shield housing 124 (FIGS. 1-5), however, an opening 630 may be slightly radially lengthened, if necessary, to provide room for an innermost needle blocking object 622A and an outermost abutting needle blocking object 622B, i.e., a pair of adjacent blocking objects. (Note, outermost blocking object 622B may or may not actually block needle 102.) Both blocking objects are preferably ball bearings. This lengthened opening 630 may be accomplished by extending a flange 637 compared to that of FIGS. 1-5. The purpose behind this embodiment is that previously described embodiments (FIGS. 1-5 and 8-16) may in some cases expose their respective biasing member to sharp distal end 108 of needle 102 in the blocking position. Although extremely unlikely, this situation may lead to failure of the respective biasing member and possible movement of the needle blocking object such that sharp distal end 108 can emerge from the needle shield housing. Needle shield housing 624 of this embodiment addresses this potential by accommodating two blocking objects 622A, 622B such that a biasing member 632 cannot come into contact with sharp distal end 108 of needle 102. Further, sharp distal end 108 cannot emerge from needle shield housing 624 even if an innermost needle blocking object 622A becomes loose, e.g., proximally of an outermost blocking object 622B and within any of the lumens that may be provided. In this case, blocking objects 622A, 622B would together always limit distal movement of needle 102 so that it cannot emerge from needle shield housing 624. The cost of this embodiment is not significantly greater because the costs of blocking objects 622A, 622B are minimal.

Operation of needle shield housing 624 will be described relative to an embodiment including telescoping member 120. It is noted, however, that the FIGS. 15-16 embodiment is combinable with this two blocking object embodiment. In operation, blocking objects 622A, 622B both move by exertion of biasing force F towards the blocking position (FIG. 18), preventing needle 102 from emerging from needle shield housing 624, in response to a triggering proximal movement of the needle. A triggering proximal movement of the needle occurs in this case when needle 102 moves proximally relative to needle shielding housing 624 sufficiently to engage telescopic member 120 and move telescopic member 120 out of contact with innermost blocking object 622A. In particular, as needle 102 moves proximally, stopping feature 107 (FIG. 1) of sharp distal end 108 (such as the bend or other radially extending structure) engages lumen 176 (FIG. 6) of telescoping member 120. As this occurs, telescoping member 120 extends longitudinally relative to lumen 626 of needle shield housing 624. As described above, this movement enlarges the volume of lumen 626 and thus needle shield housing 624 so as to accommodate sharp distal end 108, and eventually moves telescoping member 120 out of contact with innermost needle blocking object 622A. The telescopic movement of telescoping member 120 continues until flange 180 thereof engages end flange 151 of enclosure 148 (FIG. 2), which prevents separation of telescoping member 120 from enclosure 148 and needle shield housing 624. As noted above, stopping feature 107 of sharp distal end 108 prevents needle 102 from separating from telescoping member 120.

Once this triggering proximal movement occurs, both blocking objects 622A, 622B move freely towards the blocking position, shown in FIG. 18, under the influence of biasing force F. More specifically, once telescopic member 120 passes innermost needle blocking object 622A, both blocking objects 622A, 622B initially move substantially perpendicular to the longitudinal axis of needle 102 as they move along guiding surface 170 of opening 630. Blocking objects 622A, 622B are forced towards lumen 626 by the substantially perpendicular force F_{R} of biasing force F and are held in contact with guiding surface 170 by opening 630. As shown in FIG. 18, once guiding surface 170 can no longer guide innermost needle blocking object 622A towards lumen 626, innermost needle blocking object 622A is moved distally by the substantially parallel force F_{P} (relative to longitudinal axis A (FIG. 1 only)) and into nest 133, where it is held under the influence of biasing force F. In this case, biasing force F is applied by outermost needle blocking object 622B upon innermost needle blocking object 622A. The distal movement of innermost needle blocking object 622A after passing guiding surface 170 is such that outermost blocking object 622B rotates to have a contact point on a proximal side of equator E of innermost needle blocking object 622A such that the biasing force F now also has a substantially parallel component F_{P} (relative to longitudinal axis A (FIG. 1 only)) that acts distally. (In FIG. 17, the substantially parallel component F_{P} acts proximally, but is restrained to move blocking objects 622A, 622B by flange 637 and opening 630.) At this point, sharp distal end 108 of needle 102 is positioned within lumen 626 and/or telescoping member 120, and cannot move distally to emerge from needle shield housing 624. If innermost needle blocking object 622A becomes loose, e.g., moves proximal of outermost blocking object 622B, outermost blocking object 622B will move more into lumen 626 such that no distal movement of needle 102 is possible. During transition from the non-blocking position (FIG. 17) to the blocking position (FIG. 18), biasing member 632 is guided by a guide 650 so as to maintain contact with outermost blocking object 622B and thus maintain the appropriate direction of biasing force F. Once in the blocking position, biasing member 632 may be held by a recess 652 (FIG. 18) at an inner end of guide 650. Biasing member 632 is preferably restricted from entering lumen 626 so that needle 102 cannot come into contact and possibly damage or dislodge biasing member 632.

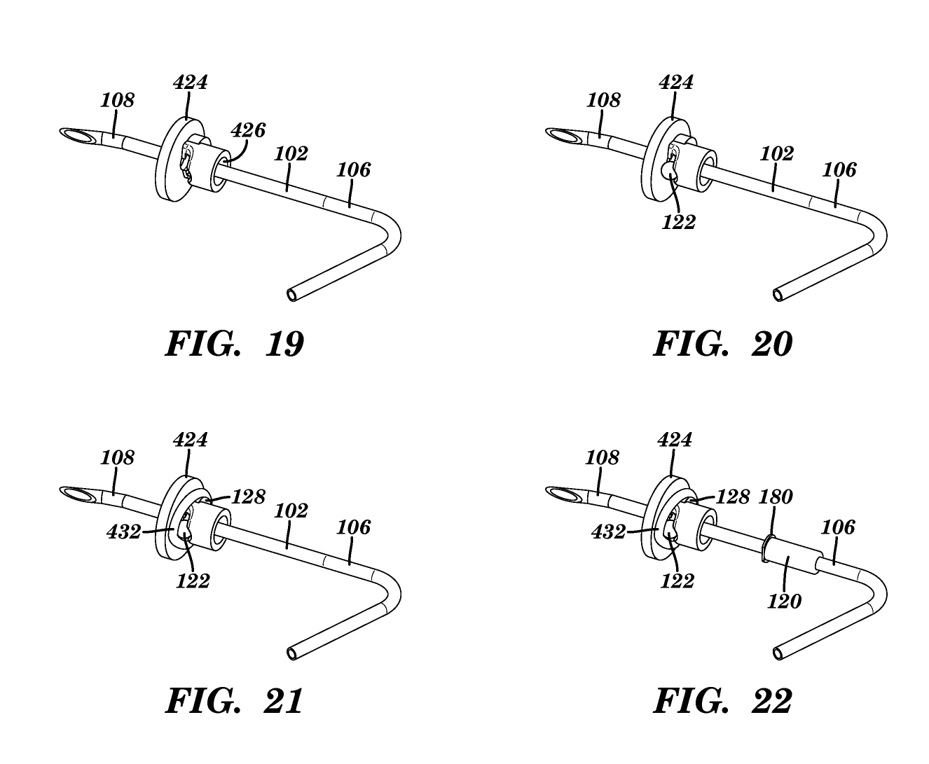

Referring now collectively to FIGS. 19-27, embodiments of a method of constructing needle-based medical device are illustrated. The embodiments of the method described herein includes telescopic member 120 per the embodiment of FIGS. 9-14. However, it is not intended to limit the method of constructing a needle-based medical device to that particular embodiment. The methods of constructing may also apply to any of the embodiments described herein with or without telescopic member 120 and with the biasing member proximal or distal of equator E.

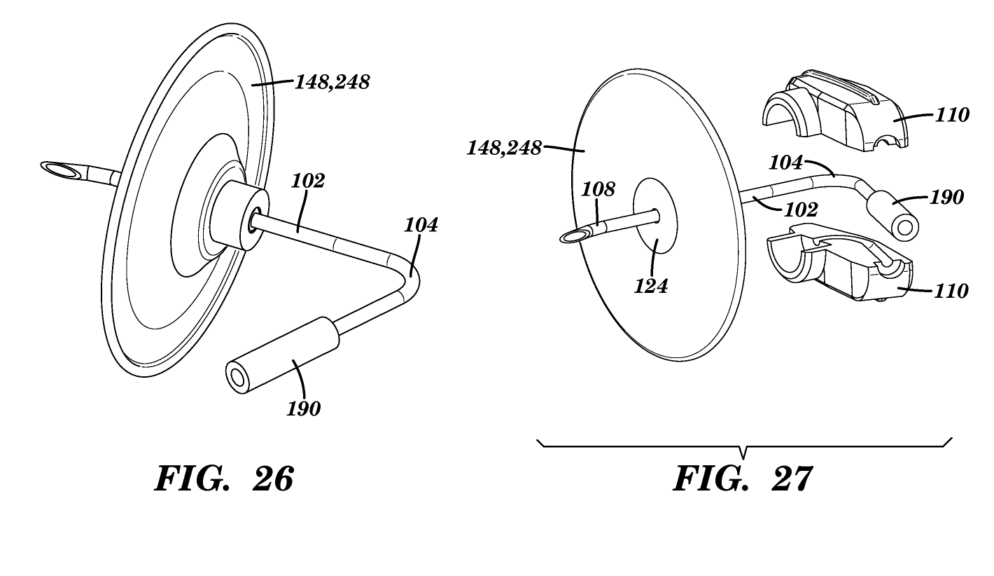

At the outset, it is also emphasized that while the figures are illustrated using a needle 102 already including a bend 104, it is possible, and probably easier, to proceed with the following processes (up to, but not including FIG. 26), and then create bend 104 in needle 102. It is also understood that the processes described herein are conducted by placing parts onto needle 102 from proximal end 106, so as to lower the possibility of damaging sharp distal end 108.

FIG. 19 shows a first step of placing needle shield housing 124 onto needle 102 such that lumen 426 of needle shield housing 424 surrounds at least a part of needle 102. This step may be carried out by threading needle 102 into lumen 426 (not shown in FIG. 19), and through the smaller diameter lumen adjacent to nest 133 (FIG. 12). Next, referring to FIG. 20, needle blocking object 122 is positioned at least partially within opening 430 (FIG. 12) through a wall of needle shield housing 424 in the non-blocking position. Needle 102 prevents needle blocking object 122 from moving too far into lumen 426 (FIG. 12). Where the FIGS. 17-18 embodiment is employed, it is understood that innermost blocking object 622A and abutting, outermost blocking object 622B may be positioned at this time.

Referring to FIG. 21, positioning a biasing member 132 for applying a biasing force on needle blocking object 122 is next. The embodiment shown includes an O-ring; however, other embodiments (FIGS. 7A-7G) may be employed. As noted above, and shown in FIGS. 12-14, biasing force F includes a first component F_{R} substantially perpendicular to the longitudinal axis of the needle and a second component F_{P} substantially parallel to the longitudinal axis of the needle. Where cam 128 is separate from needle shield housing 424, it may also be positioned adjacent to needle shield housing 424 for stretching biasing member 432 at this point, e.g., by stretching biasing member 432 sufficiently to make room for cam 128 and then releasing the biasing member to hold the cam in position.

FIG. 22 shows placing telescopic member 120 around needle 102 and into needle shield housing 424, such that flange 180 (FIG. 12) enters lumen 426 of needle shield housing 424 first. Flange 180 may be configured to abut an end flange 151 (FIG. 10) of enclosure 148 in the needle-blocking position. As noted above, flange 180 may include cut-away 182 to allow for passing of telescoping member 120 past needle-blocking object 122 so as to allow its movement substantially perpendicular to an axis of needle shield housing 124. Cut-away 182 may be positioned to be adjacent to needle-blocking object 122 upon insertion, which also aids in telescopic member 120 insertion past needle blocking object 122.

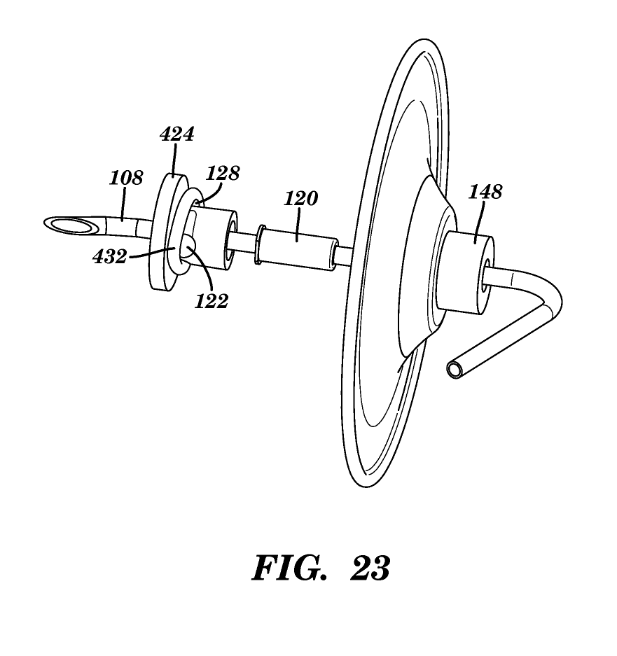

Referring now to FIG. 23, enclosure 148 is now attached to needle shield housing 424 such that enclosure 148 substantially surrounds needle shield housing 424. In an alternative embodiment not including telescopic member 120, the method may further include inserting stop-washer 584 (FIGS. 15-16) into an end of enclosure 548 (FIGS. 15-16). As mentioned above in reference to FIGS. 15-16, stop-washer 584 is configured to prevent removal of needle 102. In the blocking position, stop-washer 584 may prevent complete removal of needle 102 from lumen 426 by engaging the bend or other radial extension of sharp distal end 108. Stop washer 584 is held in position by engagement with end flange 151, or perhaps by an interference fit, where necessary.

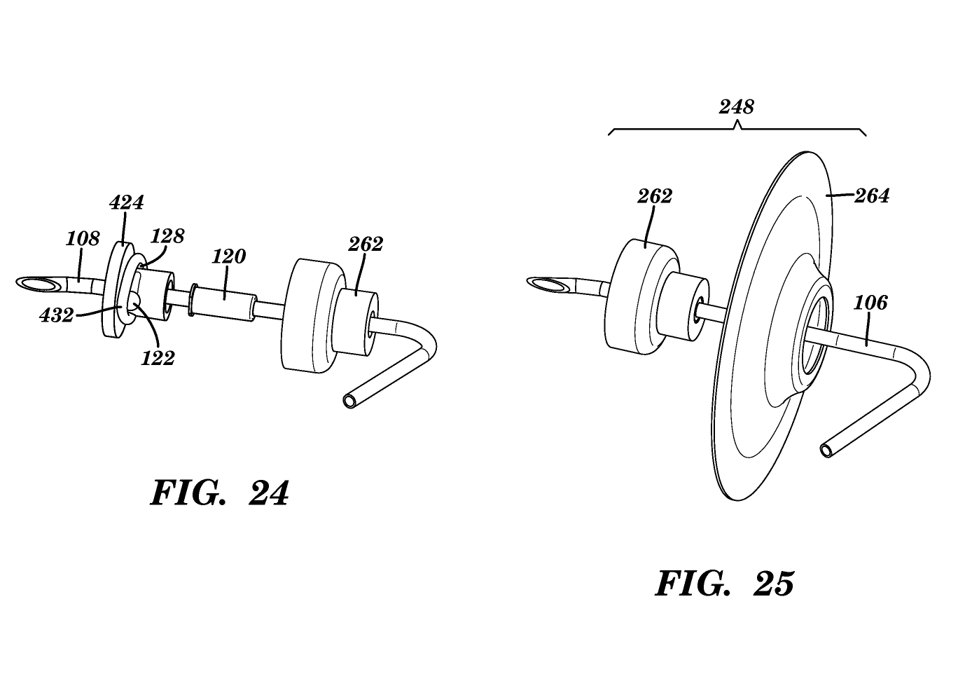

Referring now to FIGS. 24-25, an alternative embodiment of enclosure 248, as shown in FIG. 8, is configured to attach to and substantially surround needle shield housing 424. In this embodiment, inner portion 262 may include a rigid material, as discussed in detail above. Once inner portion 262 is attached to needle shield housing 424 and substantially surrounds needle shield housing 424, disk 264 may be secured to inner portion 262. Disk 264 may include a soft, flexible material, as discussed in detail above.

After enclosure 148, 248 is attached to needle shield housing 424, as shown in FIG. 26, an extension tube 190 may be attached to proximal end 106 of needle 102. At this point, if needle 102 has not been bent to include bend 104, that process may be carried out using conventional techniques. Finally, as shown in FIG. 27, needle hub 110 may be attached to needle 102 and extension tube 190. As illustrated, needle hub 110 includes two separate snap together parts; however, other processes may also be employed to form needle hub 110 such as injection molding, and other hub configurations may be used.

The terms “first,” “second,” and the like, herein do not denote any order, quantity, or importance, but rather are used to distinguish one element from another, and the terms “a” and “an” herein do not denote a limitation of quantity, but rather denote the presence of at least one of the referenced item. The modifier “about” used in connection with a quantity is inclusive of the stated value and has the meaning dictated by the context, (e.g., includes the degree of error associated with measurement of the particular quantity) and by the knowledge of persons of ordinary skill in the art. The suffix “(s)” as used herein is intended to include both the singular and the plural of the term that it modifies, thereby including one or more of that term (e.g., the metal(s) includes one or more metals).

The foregoing description of various aspects of the invention has been presented for purposes of illustration and description. It is not intended to be exhaustive or to limit the invention to the precise form disclosed, and obviously, many modifications and variations are possible. Such modifications and variations that may be apparent to an individual in the art are included within the scope of the invention as defined by the accompanying claims.

1-47. (canceled)

48. A medical device comprising:
a needle having a proximal end, a sharp distal end and a longitudinal axis;
a needle blocking object movable from a non-blocking position offset from the longitudinal axis of the needle to a blocking position;
a needle shield housing, the needle shield housing including:
a lumen for accommodating at least part of the needle such that the needle shield housing is slidable at least partially along the longitudinal axis of the needle;
an opening for accommodating at least part of the needle blocking object in the non-blocking position; and
a biasing member for applying a biasing force on the needle blocking object, the biasing force including a first component substantially perpendicular to the longitudinal axis of the needle and a second component substantially parallel to the longitudinal axis of the needle,
wherein the needle blocking object moves by exertion of the biasing force to the blocking position, preventing the needle from emerging from the needle shield housing, in response to a triggering proximal movement of the needle.

49. The medical device of claim 48, wherein the biasing member substantially surrounds the needle, the biasing member comprises a generally circular elastic member, and the generally circular elastic member is substantially coaxial with the longitudinal axis.

50. The medical device of claim 48, further comprising an enclosure for covering the needle shield housing, wherein the biasing member is on the outside of the needle shield housing.

51. The medical device of claim 50, wherein the enclosure further comprises a disk extending away from the needle shield housing.

52. The medical device of claim 48, further including a guide for the biasing member to substantially maintain the biasing member in position relative to the needle blocking object.

53. The medical device of claim 52, wherein the guide comprises a bearing surface,
wherein the bearing surface is at least partially one of:
substantially perpendicular to the longitudinal axis,
at an acute angle relative to the longitudinal axis, and
at an obtuse angle relative to the longitudinal axis; and
wherein the bearing surface is in communication with the opening.

54. The medical device of claim 50, wherein the guide includes a recessed portion for receiving at least part of the biasing member, such that at least one part of the biasing member is closer to the longitudinal axis of the needle than another part of the biasing member in the blocking position.

55. The medical device of claim 48, wherein the needle blocking object is a ball bearing.

56. The medical device of claim 55, wherein the ball bearing includes an equator and wherein the biasing member exerts the biasing force on a proximal side of the equator.

57. The medical device of claim 55, wherein the ball bearing includes an equator and wherein the biasing member exerts the biasing force on a distal side of the equator in the non-blocking position and transitions to exert the biasing force on a proximal side of the equator during transitioning from the non-blocking position to the blocking position.

58. The medical device of claim 48, wherein the needle has a stopping feature adapted to interact with part of the needle shield housing to prevent distal movement of the needle shield housing when the needle blocking object is in the blocking position.

59. The medical device of claim 58, wherein the triggering proximal movement includes movement of the sharp distal end of the needle out of contact with the needle blocking object.

60. The medical device of claim 48, further comprising an enclosure for covering the needle shield housing, the enclosure including another lumen aligned with the lumen of the needle shield housing.

61. The medical device of claim 48, wherein the needle is a Huber needle.

62. The medical device of claim 48, wherein the needle blocking object includes an innermost needle blocking object and an abutting, outermost needle blocking object, and
wherein the biasing member applies the biasing force on the outermost needle blocking object and the innermost needle blocking object moves by exertion of the biasing force to the blocking position.

63. A method of constructing a needle-based medical device, the method comprising:
placing a needle shield housing around a needle such that a lumen of the needle shield housing surrounds at least a part of the needle;
positioning a needle blocking object at least partially within an opening in the needle shield in a non-blocking position; and
positioning a biasing member for applying a biasing force on the needle blocking object, the biasing force including a first component substantially perpendicular to the longitudinal axis of the needle and a second component substantially parallel to the longitudinal axis of the needle.

64. The method of claim 63, further comprising securing a disk to the needle based medical device.

65. The method of claim 63, wherein the positioning of the needle blocking object includes positioning an innermost needle blocking object and an outermost blocking object.

66. The method of claim 65, wherein the outermost blocking object is placed in abutment with the innermost needle blocking object.

67. A medical device comprising:
a needle having a proximal end, a sharp distal end and a longitudinal axis;
a ball bearing movable from a non-blocking position offset from the longitudinal axis of the needle to a blocking position;
a needle shield housing, the needle shield housing including:
a lumen for accommodating at least part of the needle such that the needle shield housing is slidable at least partially along the longitudinal axis of the needle;
an opening for accommodating at least part of the ball bearing in the non-blocking position; and
a biasing member for applying a biasing force on the ball bearing, the biasing force including a first component substantially perpendicular to the longitudinal axis of the needle and a second component substantially parallel to the longitudinal axis of the needle,
wherein the ball bearing moves by exertion of the biasing force to the blocking position, preventing the needle from emerging from the needle shield housing, in response to a triggering proximal movement of the needle, and
wherein the ball bearing is retained in the blocking position by the biasing force.
